(19) United States
(12) Patent Application Publication
(10) Pub. No.: US 2022/0218952 A1
(43) Pub. Date: Jul. 14, 2022

(54) DEVICE AND SYSTEM FOR LOCATING GUIDEWIRE

(71) Applicant: MEDSTAR HEALTH, INC., Columbia, MD (US)

(72) Inventor: Gajan Sivananthan, Washington, DC (US)

(21) Appl. No.: 17/571,729

(22) Filed: Jan. 10, 2022

Related U.S. Application Data
(60) Provisional application No. 63/135,774, filed on Jan. 11, 2021.

(51) Int. Cl. A61M 25/02; A61M 25/09

(52) U.S. Cl. CPC A61M 25/02; A61M 2025/024; A61M 2025/0266; A61M 25/09

(57) ABSTRACT

A device comprises a base configured to be secured to a support surface within a surgical field. A top tab is spaced above the base and extends inwardly from one end thereof. A bottom tab is spaced above the base and extends inwardly from an opposite side of the base relative to the top tab. An end portion of the bottom tab underlies the top tab and defines a gap therebetween. The bottom tab is movable between a first position where the gap is sufficiently closed to prevent the passage of a guidewire therethrough, and a second position where the gap is sufficiently open to permit the passage of the guidewire therethrough. An opening is defined between the base and the top and bottom tabs to locate the guidewire and allow longitudinal movement therethrough.

### CROSS-REFERENCE TO PRIORITY APPLICATION

This application claims priority under 35 U.S.C. § 119 to U.S. Provisional Patent Application No. 63/135,774, filed Jan. 11, 2021, entitled “Guidewire Guards,” in the name of Gajan Sivananthan, which is hereby expressly incorporated by reference in its entirety as part of the present disclosure.

### FIELD OF THE INVENTION

The present invention relates to devices and systems for locating guidewires, and more particularly, to devices and systems for locating guidewires within sterile surgical fields.

### BACKGROUND INFORMATION

A guidewire is a device used to enter tight spaces, e.g., obstructed channels, within the body, or to assist in inserting, positioning, and moving a catheter. Guidewires vary in size, length, stiffness, composition, and shape of the tip, but are typically long, thin, flexible wires. A catheter is a tubular medical device for insertion into canals, vessels, passageways, or body cavities usually to permit injection or withdrawal of fluids or to keep a passage open. A cannula is a tube or sheath inserted into a cavity to serve as a channel for the transport of fluid either into or out of the body. A cannula may enclose a trocar. After such a cannula is inserted into a blood vessel, body cavity, duct, or hollow organ, withdrawal of the trocar lets fluid drain (so that it can be collected or sampled) or escape. Tubular medical devices, such as catheters or cannulas, are used with guidewires by introducing a guidewire into a body, such as through a sharp hollow needle. The guidewire is advanced through the lumen of the needle to a desired position, such as within a cavity or vessel, and then the needle may be withdrawn. Like guidewires, the tubular medical devices used with guidewires are also long, slender, flexible tubular devices defining tubular cavities or lumens therethrough for receiving guidewires and for allowing the tubular medical devices to be passed over the guidewires. A tubular medical device, such as a catheter or cannula, is passed over the guidewire and into the cavity or vessel. After passing the tubular medical device, the guidewire may be withdrawn.

Guidewires and their associated tubular medical devices, such as catheters and cannulas, are sterile and their sterility must be preserved prior to introduction into the body. Many endovascular and minimally invasive procedures involve using long guidewires and catheters, and such guidewires, catheters and other tubular medical devices are prone to fall off the sterile field of a surgical table or platform. If one end of a guidewire or other tubular device falls off the sterile field, it can cause a risk of infection if not noticed—even if that end of the guidewire is not inserted into the patient, a tubular device passed over the guidewire can be similarly contaminated, increasing the risk of infection. When the contamination is noticed, the contaminated guidewire or other device must be discarded and a new guidewire or other device may need to be inserted, increasing the time and cost of the procedure.

Often, to prevent contamination, a person's hand will be used to ensure that the guidewires and catheters and other tubular medical devices remain in the proper location. This may be the hand of a member of the surgical team, or a separate technician may be assigned the task. If a member of the surgical team performs this task, his or her hand is then unavailable to perform other tasks. On the other hand, assigning the task to a technician adds another person in a limited working space. There can be a high cost to this solution. In addition, requiring a physician to hold the guidewire(s) in place can be a distraction and otherwise take his or attention away from other tasks which can lead to reduced efficiencies.

An alternate solution is to use towels to hold the guidewires in place. Towels may be placed on top of the guidewires to hold them in place when they are not actively being manipulated. These towels may be wet to make them heavier and more effective at preventing unwanted movement of the guidewires. However, the towels may shed fibers onto the guidewires, and those fibers may be displaced into the body when the guidewire is advanced into the body or when a tubular device is advanced along the guidewire such that at least some fibers pass from the guidewire onto the tubular device as it moves along the guidewire. These fibers, if introduced into the body, can increase the risk of complications. In addition, the towels must be moved off the guidewires in order to move the guidewires or tubular medical devices over the guidewires. A towel, similar to the guidewires themselves, could fall off the sterile filed, increasing the risk of contamination if not noticed or causing delay and increased cost.

Surgical instruments such as forceps may be used in lieu of wet towels to locate or otherwise manage guidewires in a sterile surgical field. However, forceps must be removed each time a tubular medical device is advanced over the guidewire. Further, the forceps can kink or otherwise damage the guidewire, potentially inhibiting proper operation of the guidewires, and the forceps can be slippery, potentially themselves falling off the sterile field.

In addition, a physician may be required to redirect a guidewire to put the far end in a more useful or ergonomic location, such as towards the center of a surgical field. For example, a guidewire pointed toward the face of a patient may be redirected back toward the surgical site, both to prevent interaction between the guidewire and the face of the patient and to ensure easier access to the guidewire. Such redirection may be performed by a human holding the device or by using a towel or forceps to hold the guidewire down, and thereby can pose the same or similar drawbacks or disadvantages as described above.

The above-described drawbacks and disadvantages can be exacerbated when using multiple guidewires. If a towel is used to hold down multiple guide wires, particularly redirected guidewires, the medical team must be careful to ensure that none of the guidewires fall off the sterile field if, for example, one of the guidewires must be removed from under the towel to be manipulated. On the other hand, if a physician or technician is holding the guidewires, keeping track of the respective guidewires can be a logistical challenge and can take the focus of that person away from other tasks. Accordingly, the foregoing solutions of the prior art are makeshift and can pose significant drawbacks and disadvantages.

It is an object of the present invention, and/or of embodiments thereof, to overcome one or more of the above-described drawbacks and/or disadvantages of the prior art.

### SUMMARY OF THE INVENTION

In accordance with a first aspect, the present invention is directed to a device for locating a guidewire and/or tubular medical device within a field associated with a surgical procedure, such as a sterile surgical or operating field, and for maintaining sterility of the guidewire and/or tubular medical device in connection with the surgical procedure. The guidewire and/or tubular medical device defines a longitudinal direction of movement. The device comprises a base configured to be secured to a support surface within the field, such as a sterile drape, towel, or table, to thereby define a position of the device within the field. A top or upper tab is spaced above the base and extends inwardly from one end thereof. A bottom or lower tab is spaced above the base and extends inwardly from a substantially opposite side of the base relative to the top tab. At least a portion of the bottom tab underlies the top tab and defines a gap therebetween. At least one of the top tab and/or the bottom tab is movable between a first position and a second position. In the first position, the gap is sufficiently closed to prevent the passage of the guidewire and/or tubular medical device therethrough. In the second position, the gap is sufficiently open to permit the passage of the guidewire and/or tubular medical device therethrough. An opening of the device is defined between the base and the top and bottom tabs. The opening is configured to allow the guidewire and/or tubular medical device to be received therein to locate the guidewire and/or tubular medical device at the position of the device within the field and allow movement of the guidewire and/or tubular medical device in the longitudinal direction of movement within the opening.

In some embodiments of the present invention, the top tab has an inner face and an outer face and the bottom tab has an inner face and an outer face. The top tab overlaps the bottom tab, and the gap is defined between the overlapping inner face of the top tab and outer face of the bottom tab. In some such embodiments, the bottom tab is depressible relative to the top tab between the first and second positions. In some such embodiments, the top tab is not movable between the first and second positions, but rather is immovable or fixed on the device.

In some embodiments of the present invention, the bottom tab is normally biased or urged in a direction from the second position toward the first position. In some such embodiments, the guidewire and/or tubular medical device is receivable within the gap and is engageable between the bottom and top tabs to secure the guidewire and/or tubular medical device therein and substantially prevent longitudinal movement thereof. In some embodiments of the present invention, at least the overlapping portions of the inner face of the top tab and the outer face of the bottom tab are substantially smooth to facilitate depressing the bottom tab with the guidewire and/or tubular medical device relative to the top tab, and passing the guidewire and/or tubular medical device through the gap and into the opening to locate the guidewire and/or tubular medical device at the position of the device within the field and allow longitudinal movement of the guidewire and/or tubular medical device within the opening. Preferably, the bottom tab is normally biased or urged in a direction towards the top tab such that the overlapping portions of the top tab and bottom tab are closely spaced or in contact with each other, thereby minimizing or eliminating the gap between the overlapping portions of the tabs.

Some embodiments of the present invention further comprise a first or inner arch extending between the top tab and the base, and a second or outer arch extending between the bottom tab and the base. In some such embodiments, the top tab defines a first length and the bottom tab defines a second length that is greater than the first length. In some embodiments, the first arch is oriented at first angle relative to the base, the second arch is oriented at a second angle relative to the base, and the second angle is greater than the first angle. In some embodiments of the present invention, the first arch defines a first joint between the first arch and the base, and a second joint between the first arch and the bottom tab. The first and/or second joints are configured to allow the bottom tab to flex about the respective joint to thereby allow the bottom tab to move between the first and second positions. In some embodiments of the present invention, the second joint is configured to allow the bottom tab to be depressed in response to a downward force exerted thereon and flexed about the second joint from the first position to the second position, and to normally bias or urge the bottom tab from the second position to the first position after release of the downward force therefrom. In some embodiments of the present invention, the first joint is configured to allow the bottom tab to be depressed in response to a downward force exerted thereon and flexed about the first joint from the first position to the second position, and to normally bias or urge the bottom tab from the second position to the first position after release of the downward force therefrom. Preferably, the device is formed of plastic that allows the bottom tab to flex between the first and second positions but normally biases or urges the bottom tab toward the first or closed position.

In some embodiments of the present invention, the second arch defines a first joint between the second arch and the base, and a second joint between the second arch and the top tab. Preferably, the first and second joints are configured to substantially prevent movement of the top tab relative to the bottom tab or base, i.e., the position of the top tab is fixed or immovable on the device.

In some embodiments of the present invention, the device further comprises within the opening a relatively raised surface and a relatively recessed surface contiguous to the raised surface. The relatively recessed surface is configured to receive and releasably retain therein the guidewire and/or tubular medical device and substantially prevent longitudinal movement thereof. Some such embodiments further comprise a plurality of the relatively raised surfaces and relatively recessed surfaces. Each relatively recessed surface is configured to receive and releasably retain therein a respective guidewire and/or tubular medical device and substantially prevent longitudinal movement thereof. In some embodiments of the present invention, the relatively raised surface and relatively recessed surface define a groove or channel formed on or at the base and extending longitudinally in a direction from one side of the base to an opposite side of the base. In some embodiments of the invention, the relatively raised surface and the relatively recessed surface define a groove or channel formed on or at the inner face of the top tab or on or at the inner face of the bottom tab, or on another interior surface of the device, and extending longitudinally in a direction from one side of the device to the other side of the device.

Some embodiments of the present invention further comprise an adhesive, such as a pressure-sensitive adhesive, on an underside of the base configured for adhesively attaching the base to the support surface and securing the device thereto.

In accordance with another aspect, the present invention is directed to a system for locating one or more guidewires and/or tubular medical devices within a field associated with a surgical procedure and for maintaining sterility of the guidewire and/or tubular medical device in connection with the surgical procedure. The system comprises a plurality of devices as described above, wherein each device is locatable at a respective position within the field. In some embodiments of the present invention, a first device defines a first elongated axis and is engageable with the support surface at a first position within the field, and a second device defines a second elongated axis and is engageable with the support surface at a second position within the field spaced relative to the first position. The second elongated axis is oriented at an acute angle or greater relative to the first elongated axis for positioning and guiding the guidewire and/or tubular medical device along a curved path. In some such embodiments, in the second position, the second elongated axis is substantially perpendicular to the first elongated axis in the first position. Some embodiments of the present invention further comprise a third device defining a third elongated axis and engageable with the support surface on a substantially opposite side of the first device relative to the second device for locating and guiding the guidewire and/or other tubular medical between the third device and first device. In some such embodiments, the third device substantially prevents a proximal end of the guidewire and/or other tubular medical from falling outside of the field, and the first and second devices locate and guide the guidewire and/or other tubular medical toward an entry point for the surgical procedure.

In accordance with another aspect, the present invention is directed to a device for locating a guidewire and/or tubular medical device within a field associated with a surgical procedure and for maintaining sterility of the guidewire and/or tubular medical device in connection with the surgical procedure. The guidewire and/or tubular medical device defines a longitudinal direction of movement. The device comprises first means for securing the device to a support surface within the field and defining a position of the device within the field. The device further comprises second means spaced above the first mean and extending inwardly from one end thereof, and third means spaced above the first means and extending inwardly from a substantially opposite side of the first means relative to the second means. At least a portion of the third means underlies the second means and defines a gap therebetween. The second means and/or the third means is for moving between a first position and a second position. In the first position, the gap is sufficiently closed to prevent the passage of the guidewire and/or tubular medical device therethrough. In the second position, the gap is sufficiently open to permit the passage of the guidewire and/or tubular medical device therethrough. An opening is defined between the first means and the second and third means. The opening is configured to allow the guidewire and/or tubular medical device to be received therein to locate the guidewire and/or tubular medical device at the position of the device within the field and allow movement of the guidewire and/or tubular medical device in the longitudinal direction of movement within the opening.

In some embodiments of the present invention, the first means is a base, the second means is a top tab, and the third means is a bottom tab. Some embodiments of the present invention further comprise fourth means for attaching the first means to the support surface. In some such embodiments, the fourth means is a layer of adhesive.

In accordance with another aspect, the present invention is directed to a method for locating a guidewire within a field associated with a surgical procedure and for maintaining sterility of the guidewire in connection with the surgical procedure. The method comprises the following steps:

(i) securing a base of a device to a support surface within the field and defining a position of the device within the field;

(ii) engaging a lower tab of the device with the guidewire, depressing the lower tab relative to an upper tab of the device, and opening or expanding a gap between the lower and upper tabs;

(iii) sliding the guidewire through the opened or expanded gap;

(iv) allowing the lower tab to move back toward the upper tab to close or reduce the gap between the lower and upper tabs to prevent the passage of the guidewire therethrough; and

(v) receiving the guidewire in an opening of the device formed between the tabs and the base of the device to locate the guidewire at the position of the device within the field and thereby maintain sterility of the guidewire, and allow movement of the guidewire in a longitudinal direction of movement of the guidewire within the opening in connection with the surgical procedure.

In some embodiment of the present invention, the securing step includes pressing an adhesive-backed surface of the base against the support surface to secure the base to the support surface and define the position of the device within the field. Some embodiments of the present invention further comprise positioning a plurality of devices spaced relative to each other within the field to define a guidewire path, and guiding the guidewire through the plurality of devices along the guidewire path.

One advantage of the present invention and/or of embodiments thereof, is that the device, system or method can overcome the above-described difficulties encountered in managing guidewires and associated tubular medical devices in an operative or surgical field. Each device is configured to be secured, such as by an adhesive base, to a support surface within the field, such as a sterile drape, towel, or table, to thereby define a position of the device within the field. In addition, the opening in the device is configured to allow movement of the guidewire and/or tubular medical device in the longitudinal direction of movement within the opening. As a result, each device can locate the guidewire and/or tubular medical device at the position of the device within the operative or surgical field, and allow longitudinal movement of the guidewire and/or tubular medical device to thereby guide the movement of the guidewire and/or tubular medical device. Accordingly, the problems encountered in the above-described prior art, including guidewires falling outside of the surgical or operative field, such as by falling off a patient or table supporting a patient and hitting the floor, or otherwise becoming contaminated or subjected to an unacceptable risk of contamination, can be substantially avoided.

Yet another advantage of the present invention and/or of embodiments thereof, is that the device, system or method can obviate the need for a physician, technician, or other personnel to dedicate a hand to holding each guidewire within the surgical or operative field to locate and guide the guidewire, and prevent the guidewire from falling or otherwise becoming contaminated, as encountered in the above-described prior art. Rather, each guidewire can be located within the surgical or operative field, guided within the field, and prevented from falling outside the field or otherwise being subjected to contamination, by one or more devices or system of the present disclosure. As a result, the device, system or method can free-up the hand of the physician, technician, or other personnel required by the above-described prior art to locate, guide and prevent contamination of guidewires, thereby allowing for reduced costs and/or increased attention or improved focus on other tasks.

Yet another advantage of the present invention, and/or of embodiments thereof, is that the device, system or method can obviate the need to use wet towels, or surgical instruments, such as forceps, to locate guidewires within surgical or operative fields. Rather, the guidewire is located at the location of the device within the surgical or operative field, and therefore eliminates the need so use wet towels or surgical instruments to attempt to perform this function. In addition, the problems encountered with towels shedding fibers onto guidewires, or surgical instruments, such as forceps, kinking or damaging guidewires, can be avoided. A still further advantage is that the device, system or method of the present disclosure eliminates the need to remove and/or replace a locating object as used in the above-described prior art, such as a wet towel or surgical instrument, when necessary to advance or otherwise longitudinally move the guidewire. Rather, the opening of the device is configured to define or locate a position of the device within the field and allow longitudinal movement of the guidewire through the opening.

Another advantage of the present invention, and/or of embodiments thereof, is that the device securely attaches to the sterile field, such as by an adhesive base, and securely retains the guidewire in the sterile field. As a result, the device, system or method can allow for improved operator ergonomics, such as by redirecting and routing guidewires during various medical or surgical procedures, such as endovascular surgeries or CT guided procedures. Yet another advantage is that the device, system or method allows guidewires, catheters, angioplasty balloons, stents and other tubular medical devices, to easily slide in and out through the opening of the device to thereby facilitate easy exchanges.

Another advantage of the present invention, and/or of embodiments thereof, is that the guidewire and/or tubular medical device, can be used to depress the lower tab and thereby easily slide the guidewire laterally through the gap between the lower and upper tabs and into the opening of the device. Then, once the guidewire and/or tubular medical device is slid laterally through the gap, the lower tab is normally biased or urged toward the upper tab to close or substantially close the gap and thereby retain the guidewire and/or tubular medical device within the device. A further advantage is that, if desired, a guidewire can be pinned or otherwise held between the upper tab and lower tab by laterally sliding the guidewire only partially through the gap and allowing the lower tab to engage the guidewire and pin it against the upper tab, to thereby fix the position of the guidewire and substantially prevent longitudinal movement thereof. Another advantage is the device may include within the opening a relatively raised surface and a relatively recessed surface contiguous to the raised surface, such as may be defined by a groove or channel. The relatively recessed surface is configured to receive and releasably retain therein the guidewire and/or tubular medical device and substantially prevent longitudinal movement thereof. Accordingly, if desired, the device can allow one or more guidewires to be fixed within the device to prevent longitudinal movement thereof, if and when desired.

Other advantages of the present invention, and/or of the embodiments thereof, will become more readily apparent in view of the followed detailed description of embodiments of the invention and accompanying drawings.

### BRIEF DESCRIPTION OF THE DRAWINGS

FIG. 1 is a top plan view of system including a plurality of devices embodying the present invention where each device is locatable at respective position within a sterile surgical field for redirecting a guidewire for a transradial procedure and preventing the guidewire from falling off the table or otherwise becoming contaminated during the procedure;

FIG. 2 is a top plan view of system including two sets of devices embodying the present invention for accessing a patient with two guidewires from different directions, such as for hemodialysis (“HD”) fistula thrombolysis, wherein one set of devices directs a first guidewire through a vascular sheath at one end of the fistula, and another set of devices directs a second guidewire through a vascular sheath at another end of the fistula, and where the second guidewire is redirected toward the physician;

FIG. 3 is a perspective view of one of the devices embodying the present invention of FIGS. 1 and 2;

FIG. 4 is a top plan view of the device of FIG. 3;

FIG. 5 is a bottom plan view of the device of FIG. 3;

FIG. 6 is a side elevational view of the device of FIG. 3;

FIG. 7 is a left end elevational view of the device of FIG. 6;

FIG. 8 is a right end elevational view of the device of FIG. 6; and

FIG. 9 is a side elevational view of the device of FIG. 6 showing the lower tab depressed from the first position into the second position to allow the passage of a guidewire through the gap between the upper and lower tabs and into the opening of the device.

### DETAILED DESCRIPTION OF EMBODIMENTS OF THE INVENTION

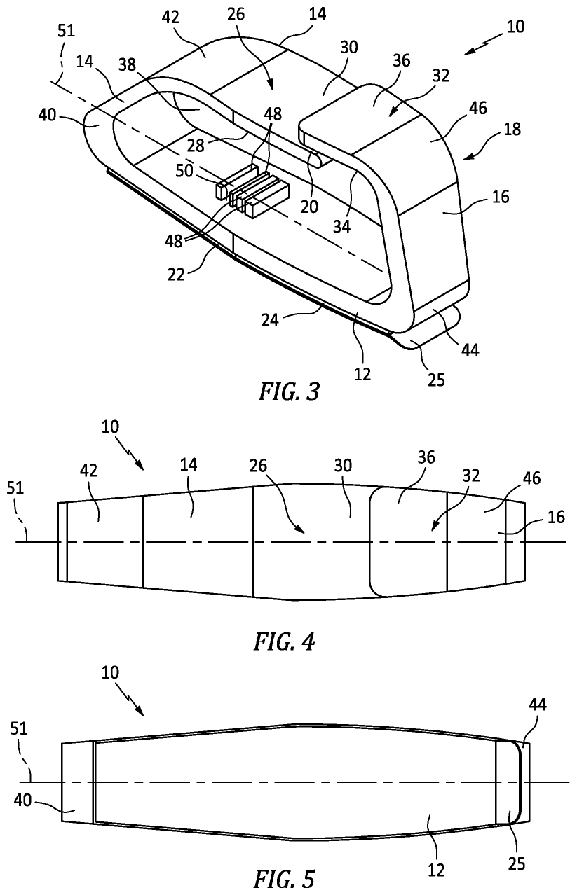

In FIGS. 3 through 9, a device indicated generally by the reference numeral 10 is configured to preserve the sterility of guidewires and improve operator ergonomics during endovascular surgeries and image guided procedures, such as computer tomography (CT) guided procedures. The device 10 can be attached to a support surface defined by, for example, a sterile drape, towel, or table, using an adhesive base 12. The device 10 includes two overlapping arches, including a first or inner arch 14 and a second or outer arch 16, to ensure that the guidewire remains secure while allowing guidewires, catheters, angioplasty balloons, and stents, for example, to easily slide in and out of a holder portion 18 of the device and thereby allow for easy exchanges of such devices therethrough. This functionality can be accomplished by the inner arch 14 which easily can be pushed down creating an opening 20 allowing devices to be secured and released as needed. In particular, the overlapping arches 14, 16 of the device 10 can meet two goals. First, the base 12 defines a bottom flat portion 22 with an adhesive 24 that can allow the device to be securely fastened to a sterile surgical field. The device 10 further includes a releasable backing 25 the overlies the adhesive layer 24 and is removed from the adhesive layer to expose the adhesive, and adhesively attached the device to a support surface. Second, the holder or top portion 18 with overlapping arches 14, 16 simultaneously can provide a closed system to secure a guidewire or other instruments within the sterile field in the intended trajectory while allowing for repositioning of the guidewire or other instrument into and out of the device when needed with only one hand by pressing down on the inner arch 14. This repositioning can be done without having to remove the device 10 from its secure position on the operating field. As such, the device 10 can be useful in redirecting the guidewire or other intervention devices allowing for optimization of user ergonomics. An additional benefit of the curved or arched configuration of the device 10 is that other items on the surgical field are less likely to get snagged on the edges. The device 10 can be used, for example, in radial artery access, antegrade femoral artery access, and dialysis access interventions.

The device 10 comprises a base 12, a depressible bottom or lower tab 26 having an inner face 28 and an outer face 30, and a top or upper tab 32 having an inner face 34 and an outer face 36. The depressible bottom tab 26 can extend from one side of the base 12 and the top tab 32 can extend from an opposing side of the base 12 and can extend in the same plane as the depressible bottom tab 26. The top tab 32 can have at least a portion with an arched configuration. In the illustrated embodiment, the arched configuration is defined by the inner arch 14. The outer face 36 of the top tab 32 can overlap the outer face 30 of the bottom tab 26. The inner face 34 of the top tab 32 and the outer face 30 of the bottom tab 26 can define the gap or channel 20 sized and dimensioned to releasably secure a guidewire. The inner face 34 of the top tab 32 can have a smooth surface. The base 12 can have a substantially flat bottom surface 22 and the bottom surface can include the adhesive 24 thereon.

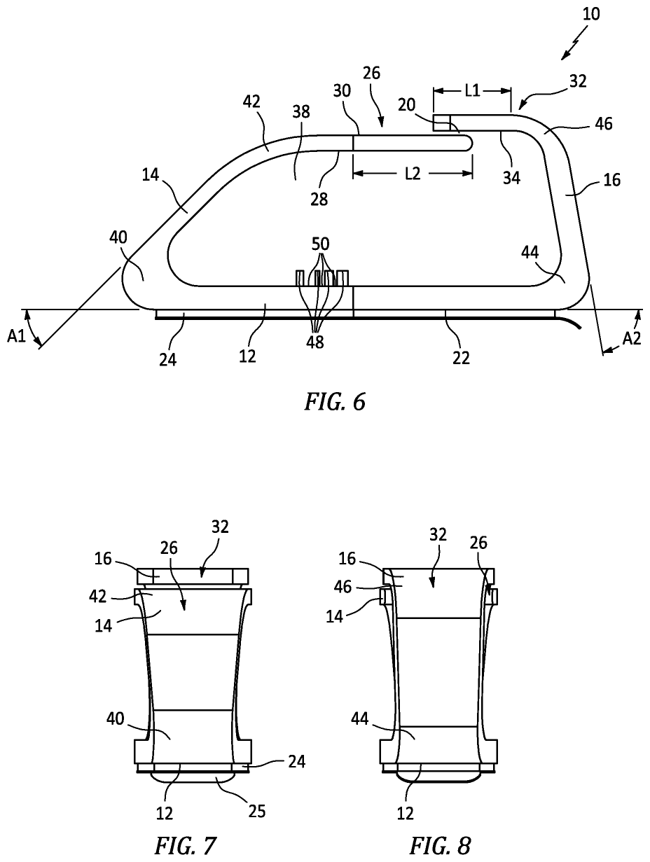

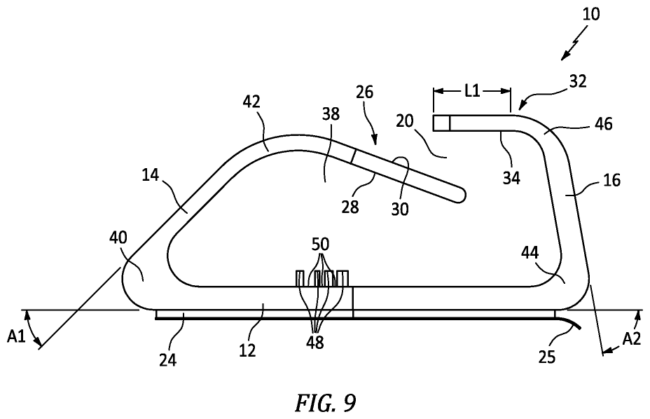

As shown typically in FIG. 9, the bottom tab 26 is movable relative to the top tab 32 between a first position (FIG. 6) and a second position (FIG. 9). In the first position, the gap 20 is sufficiently closed to prevent the passage of the guidewire and/or tubular medical device therethrough. In the second position, the gap 20 is sufficiently open to permit the passage of the guidewire and/or tubular medical device therethrough. In the illustrated embodiment, the bottom tab 26 is depressible relative to the top tab 32 between the first and second positions and the top tab 32 is fixed relative to the bottom tab 26. The bottom tab 26 is normally biased or urged in the direction from the second position (FIG. 9) toward the first position (FIG. 6). An opening 38 is defined between the base 12 and the top and bottom tabs 36 and 26, respectively. The opening 38 is configured to allow one or more guidewires and/or tubular medical devices to be received through the gap 20 between the top and bottom tabs 32 and 26, respectively, and into the opening 38 to locate the guidewire and/or tubular medical device at the position of the device within the sterile surgical or operating field, and allow movement of the guidewire and/or tubular medical device in the longitudinal direction of movement within the opening.

As shown in FIGS. 3 and 6, at least the overlapping portions of the inner face 34 of the top tab 32 and the outer face 30 of the bottom tab 26 are substantially smooth to facilitate depressing the bottom tab 26 with the guidewire and/or tubular medical device relative to the top tab 32 and passing the guidewire and/or tubular medical device through the gap 20 and into the opening 38. Once located within the opening 38, the device 10 locates the guidewire and/or tubular medical device at the position of the device within a sterile surgical or operating field and allows longitudinal movement of the guidewire and/or tubular medical device within the opening.

As shown in FIG. 6, the top tab 32 defines a first length L1 and the bottom tab 26 defines a second length L2 that is greater than the first length L1. In the illustrated embodiment, the first arch 14 is oriented at first angle A1 relative to the base 12, the second arch 16 is oriented at a second angle A2 relative to the base 12, and the second angle A2 is greater than the first angle A1. The first arch 14 defines a first joint 40 between the first arch and the base 12, and a second joint 42 between the first arch and the bottom tab 26. The first and/or second joints 40 and 42, respectively, are configured to allow the bottom tab 26 to flex about the respective joint to thereby allow the bottom tab to move between the first and second positions. In the illustrated embodiment, an as shown typically in FIG. 9, the second joint 42 is configured to allow the bottom tab 26 to be depressed in response to a downward force exerted thereon and flexed about the second joint 42 from the first position to the second position, and to normally bias or urge the bottom tab 26 from the second position to the first position after release of the downward force therefrom. If desired, the first joint 40 also may be configured to further allow the bottom tab 26 to be depressed in response to a downward force exerted thereon and flexed about the first joint from the first position to the second position, and to normally bias or urge the bottom tab from the second position to the first position after release of the downward force therefrom. The second arch 16 defines a first joint 44 between the second arch 16 and the base 12, and a second joint 46 between the second arch and the top tab 32. In the illustrated embodiment, the first and second joints 44 and 46, respectively, of the second arch 16 are configured to substantially prevent movement of the top tab 32 relative to the bottom tab 26 or base 12. The guidewire and/or tubular medical device is receivable within the gap 20, and if desired, is engageable between the bottom and top tabs 26 and 32, respectively, to secure the guidewire and/or tubular medical device therein and substantially prevent longitudinal movement thereof.

The device 10 further comprises within the opening 38 a plurality of relatively raised surfaces 48 and a relatively recessed surfaces 50 contiguous to, and between the raised surfaces 48. Each relatively recessed surface 50 is configured to receive and releasably retain therein a guidewire and/or tubular medical device and substantially prevent longitudinal movement thereof. In the illustrated embodiment, each relatively raised surface 48 and contiguous recessed surface 50 defines a respective groove or channel formed on the base 12 and extending longitudinally in a direction from one side of the base to an opposite side of the base (or substantially transverse to the elongated axis of the device). As can be seen, the grooves or channels 50 may define different widths to receive, and releasably retain therein different size guidewires or tubular medical devices. In the illustrated embodiment, the grooves or channels 50 may frictionally engage the respective guidewires to releasably retain them within the grooves, and if desired, prevent longitudinal movement of the guidewires when releasably retained therein. If desired, the device 10 may include additional grooves or channels, or the grooves or channels may be located in different positions than as shown FIGS. 3 and 6. For example, grooves or channels may be formed on the undersurface 34 of the top or upper tab 32 and/or may be formed on the undersurface 28 of the bottom tab 26. Pinning a guidewire to the undersurface of the top or bottom tab may be easier to achieve; however, the raised guidewire might be considered obtrusive. Accordingly, it may be desirable to include grooves or channels on the underside of the top and/or bottom tab and/or in or at the base, as shown. As may also be recognized by those of ordinary skill in the pertinent art based on the teachings herein, the grooves or channels may take any of numerous different shapes or configurations that are currently known, or that later become known. For example, they could be recessed within the upper surface of the base, or recessed within the undersurface of the top and/or bottom tabs. Alternatively, the device could include one or more hooks or other fasteners in the location(s) of the groove(s) for releasably retaining the guidewire within the device.

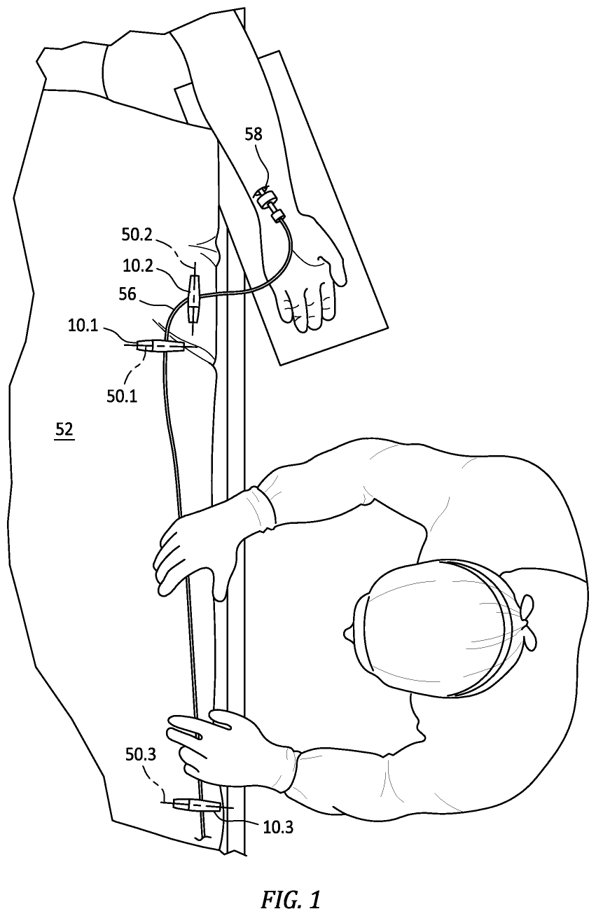

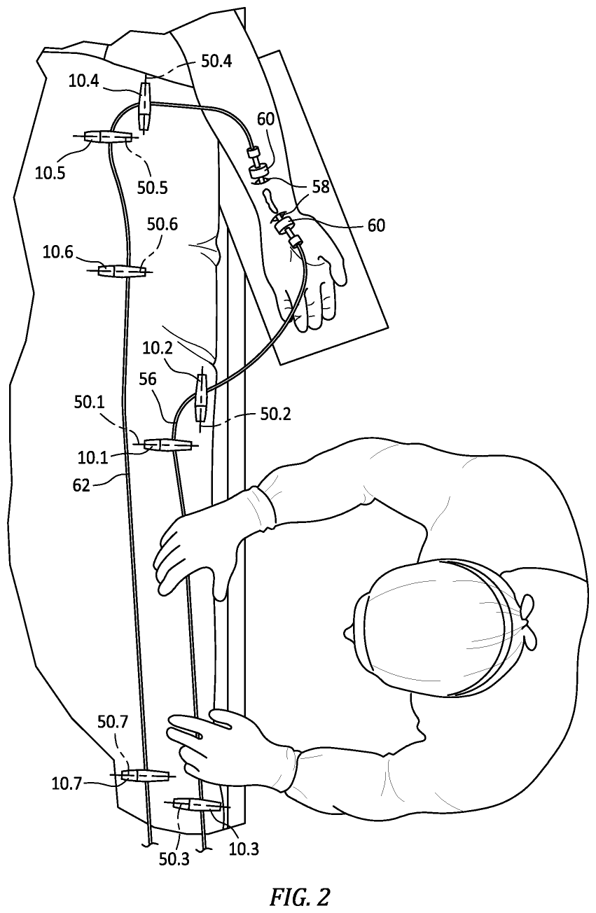

As shown typically in FIGS. 1 and 2, a system of devices 10 may be provided for locating one or more guidewires and/or tubular medical devices within a field associated with a surgical procedure and for maintaining sterility of the guidewire and/or tubular medical device in connection with the surgical procedure. The system comprises a plurality of devices 10, wherein each device 10 is locatable at a respective position within the field. Each device 10 defines an elongated axis 51. In the system, a first device 10.1 defines first elongated axis 51.1 and is engageable with the support surface 52 at a first position within the field 54, and a second device 10.2 defines a second elongated axis 51.2 and is engageable with the support surface 52 at a second position within the field spaced relative to the first position. The second elongated axis 51.2 is oriented at an acute angle or greater relative to the first elongated axis 51.1 for positioning and guiding the guidewire and/or tubular medical device along a curved path 56. In the illustrated embodiment, in the second position, the second elongated axis 51.2 in the second position is substantially perpendicular to the first elongated axis 51.1 in the first position. The system further comprises a third device 10.3 defining a third elongated axis 51.3 and engageable with the support surface 52 on a substantially opposite side of the first device 10.1 relative to the second device 10.2 for locating and guiding the guidewire and/or other tubular medical between the third device 10.3 and first device 10.1. The third elongated axis 51.3 is approximately parallel to the first elongated axis 51.1. The third device 10.3 substantially prevents a proximal end of the guidewire and/or other tubular medical from falling outside of the field, and the first and second devices 10.1 and 10.2, respectively, locate and guide the guidewire and/or other tubular medical toward an entry point or location 58 of the patient for the surgical procedure. In FIG. 1, the plurality of devices 10.1, 10.2 and 10.3 redirect a guidewire for a transradial procedure and prevent the guidewire from falling off the table or otherwise becoming contaminated during the procedure. As can be seen, the guidewire is directed into the entry location 58 and a vascular sheath 60 is received over the guidewire within the entry location.

In FIG. 2, the system includes two sets of devices for accessing a patient with two guidewires from different directions, such as for HD fistula thrombolysis, wherein one set of devices 10.1, 10.2 and 10.3 directs a first guidewire through a vascular sheath 60 at one end of the fistula (shown schematically between the access points 58, 58), and another set of devices, 10.4, 10.5, 10.6 and 10.7, directs a second guidewire through a vascular sheath 60 at another end of the fistula. The first set of devices 10.1, 10.2 and 10.3 of FIG. 2 are positioned relative to each other in substantially the same position as the devices 10.1, 10.2 and 10.3 of FIG. 1 and locate, direct, and maintain the sterility of the guidewire in the same or substantially the same manner as described above. The devices 10.4, 10.5, 10.6 and 10.7 introduce the second guidewire into a sheath 60 at the opposite end of the fistula relative to the first guidewire where the guidewire is initially directed away from the physician. Accordingly, the devices 10.4, 10.5 and 10.6, are positioned relative to the respective entry location 58 and each other to redirect the second guidewire along a curved path 62 that extends through an arc of at least about 90°. As can be seen, the elongated axis 51.4 of the device 10.4 and the elongated axis 51.5 of the device 10.5 are oriented approximately perpendicular to each other, and the elongated axes 51.5, 51.6 and 51.7 of the devices 10.4, 10.6 and 10.7, respectively, are approximately parallel to each other. Like the device 10.3 in FIG. 1, the device 10.7 of FIG. 6 substantially prevents a proximal end of the guidewire from falling outside of the field. As may be recognized by those of ordinary skill in the pertinent art based on the teachings herein, the system may include any desired number of devices 10, positioned and/or oriented relative to each other to locate one or more guidewires within a field, and to direct the guidewires along any desired path, as may be required or otherwise desired.

As may be recognized by those of ordinary skill in the pertinent art based on the teachings herein, numerous modifications, changes and/or additions may be made to the above-described and other embodiments of the present invention without departing from the scope of the invention as defined in the claims. For example, each device may include additional components or fewer components, may be made of any of numerous different materials or combinations of materials, and/or may take any of numerous different shapes and/or configurations, that are currently known or that later become known. Accordingly, the configurations and/or materials of the device(s) may be selected as dictated by the surgical procedure or other application for which the device is to be used or as may be desired. In addition, any desired number of such devices may be used together, and/or may be used with other types of devices, in any manner that is currently known, or that later becomes known, in order to locate, guide or manage guidewires or other medical devices. Accordingly, this detailed description of embodiments is to be taken in an illustrative, as opposed to a limiting sense.

What is claims is:

1. A device for locating a guidewire and/or tubular medical device within a field associated with a surgical procedure for maintaining sterility of the guidewire and/or tubular medical device in connection with the surgical procedure, wherein the guidewire and/or tubular medical device defines a longitudinal direction of movement, the device comprising:
a base configured to be secured to a support surface within the field and define a position of the device within the field;
an upper tab spaced above the base and extending inwardly from one end thereof; and
a lower tab spaced above the base and extending inwardly from a substantially opposite side of the base relative to the upper tab, wherein at least a portion of the lower tab underlies the upper tab and defines a gap therebetween, at least one of the upper tab and/or the lower tab is movable between a first position and a second position, in the first position the gap is sufficiently closed to prevent the passage of the guidewire and/or tubular medical device therethrough, and in the second position the gap is sufficiently open to permit the passage of the guidewire and/or tubular medical device therethrough, and wherein an opening is defined between the base and the upper and lower tabs, and the opening is configured to allow the guidewire and/or tubular medical device to be received therein to locate the guidewire and/or tubular medical device at the position of the device within the field and allow movement of the guidewire and/or tubular medical device in the longitudinal direction of movement within the opening.

2. A device as defined in claim 1, wherein the upper tab has an inner face and an outer face, the lower tab has an inner face and an outer face, the upper tab overlaps the lower tab, and the gap is defined between the overlapping inner face of the upper tab and outer face of the lower tab.

3. A device as defined in claim 2, wherein the lower tab is depressible relative to the upper tab between the first and second positions.

4. A device as defined in claim 3, wherein the upper tab is not movable between the first and second positions.

5. A device as defined in claim 3, wherein the lower tab is normally biased or urged in a direction from the second position toward the first position.

6. A device as defined in claim 3, wherein the guidewire and/or tubular medical device is receivable within the gap between the upper and lower tabs, and is engageable between the lower and upper tabs to secure the guidewire and/or tubular medical device therein and substantially prevent longitudinal movement thereof.

7. A device as defined in claim 2, wherein at least the overlapping portions of the inner face of the upper tab and the outer face of the lower tab are substantially smooth to facilitate depressing the lower tab with the guidewire and/or tubular medical device relative to the upper tab and passing the guidewire and/or tubular medical device through the gap and into the opening to locate the guidewire and/or tubular medical device at the position of the device within the field and allow longitudinal movement of the guidewire and/or tubular medical device within the opening.

8. A device as defined in claim 2, further comprising a first arch extending between the upper tab the base and a second arch extending between the lower tab and the base.

9. A device as defined in claim 8, wherein the upper tab defines a first length and the lower tab defines a second length that is greater than the first length.

10. A device as defined in claim 9, wherein the first arch is oriented at first angle relative to the base and the second arch is oriented at a second angle relative to the base, and the second angle is greater than the first angle.

11. A device as defined in claim 9, wherein the first arch defines a first joint between the first arch and the base, and a second joint between the first arch and the lower tab, and at least one of the first or second joints is configured to allow the lower tab to flex about the respective joint to move from the first position to the second position.

12. A device as defined in claim 11, wherein the second joint is configured to allow the lower tab to be depressed in response to a downward force exerted thereon and flexed about the second joint from the first position to the second position, and to normally bias or urge the lower tab from the second position to the first position after release of the downward force therefrom.

13. A device as defined in claim 12, wherein the first joint is configured to allow the lower tab to be depressed in response to a downward force exerted thereon and flexed about the first joint from the first position to the second position, and to normally bias or urge the lower tab from the second position to the first position after release of the downward force therefrom.

14. A device as defined in claim 9, wherein the second arch defines a first joint between the second arch and the base, and a second joint between the second arch and the upper tab, and the first or second joints are configured to substantially prevent movement of the upper tab relative to the lower tab or base.

15. A device as defined in claim 1, further comprising within the opening a relatively raised surface and a relatively recessed surface contiguous to the raised surface, wherein the relatively recessed surface is configured to receive and releasably retain therein the guidewire and/or tubular medical device and substantially prevent longitudinal movement thereof.

16. A device as defined in claim 15, further comprising a plurality of said relative raised surfaces and relatively recessed surfaces, wherein each relatively recessed surface is configured to receive and releasably retain therein a respective guidewire and/or tubular medical device and substantially prevent longitudinal movement thereof.

17. A device as defined in claim 15, wherein the relatively raised surface and relatively recessed surface defines a groove or channel formed on the base and extending longitudinally in a direction from one side of the base to an opposite side of the base.

18. A device as defined in claim 17, further comprising an adhesive on an underside of the base configured for adhesively attaching the base to the support surface and securing the device thereto.

19. A system for locating one or more guidewires and/or tubular medical devices within a field associated with a surgical procedure for maintaining sterility of the guidewire and/or tubular medical device in connection with the surgical procedure, wherein the guidewire and/or tubular medical device defines a longitudinal direction of movement, and the system comprising a plurality of devices as defined in claim 1, wherein each device is locatable at a respective position within the field.

20. A system as defined in claim 19, wherein a first device defines first elongated axis and is engageable with the support surface at a first position within the field, a second device defines a second elongated axis, is engageable with the support surface at a second position within the field spaced relative to the first position, wherein the second elongated axis is oriented at at least an acute angle relative to the first elongated axis for positioning and guiding the guidewire and/or tubular medical device along a curved path.

21. A system as defined in claim 20, wherein in the second position, the second elongated axis is substantially perpendicular to the first elongated axis in the first position.

22. A system as defined in claim 20, further comprising a third device defining a third elongated axis and engageable with the support surface on a substantially opposite side of the first device relative to the second device for locating and guiding the guidewire and/or other tubular medical between the third device and first device.

23. A system as defined in claim 21, wherein the third device substantially prevents a proximal end of the guidewire and/or other tubular medical from falling outside of the field, and the first and second devices locate and guide the guidewire and/or other tubular medical toward an entry point for the surgical procedure.

24. A device for locating a guidewire and/or tubular medical device within a field associated with a surgical procedure for maintaining sterility of the guidewire and/or tubular medical device in connection with the surgical procedure, wherein the guidewire and/or tubular medical device defines a longitudinal direction of movement, the device comprising:
first means for securing the device to a support surface within the field and defining a position of the device within the field;
second means spaced above the first mean and extending inwardly from one end thereof; and
third means spaced above the first means and extending inwardly from a substantially opposite side of the first means relative to the second means, wherein at least a portion of the third means underlies the second means and defines a gap therebetween, at least one of the second means and/or the third means is for moving between a first position and a second position, in the first position the gap is sufficiently closed to prevent the passage of the guidewire and/or tubular medical device therethrough, and in the second position the gap is sufficiently open to permit the passage of the guidewire and/or tubular medical device therethrough, and wherein an opening is defined between the first means and the second and third means, and the opening is configured to allow the guidewire and/or tubular medical device to be received therein to locate the guidewire and/or tubular medical device at the position of the device within the field and allow movement of the guidewire and/or tubular medical device in the longitudinal direction of movement within the opening.

25. A device as defined in claim 23, wherein the first means is a base, the second means is a upper tab, and the third means is a lower tab.

26. A device as defined in claim 24, further comprising fourth means for attaching the first means to the support surface.

27. A device as defined in claim 25, wherein the fourth means is a layer of adhesive.

28. A method for locating a guidewire within a field associated with a surgical procedure and for maintaining sterility of the guidewire in connection with the surgical procedure, the method comprising:
securing a base of a device to a support surface within the field and defining a position of the device within the field;
engaging a lower tab of the device with the guidewire, depressing the lower tab relative to an upper tab of the device, and opening or expanding a gap between the lower and upper tabs;
sliding the guidewire through the opened or expanded gap;
allowing the lower tab to move back toward the upper tab to close or reduce the gap between the lower and upper tabs to prevent the passage of the guidewire therethrough; and
receiving the guidewire in an opening of the device formed between the tabs and the base of the device to locate the guidewire at the position of the device within the field and maintain sterility of the guidewire, and allow movement of the guidewire in a longitudinal direction of movement of the guidewire within the opening in connection with the surgical procedure.

29. A method as defined in claim 28, wherein the securing step includes pressing an adhesive-backed surface of the base against the support surface to secure the base to the support surface and define the position of the device within the field.

30. A method as defined in claim 28, further comprising positioning a plurality of devices spaced relative to each other within the field to define a guidewire path, and guiding the guidewire through the plurality of devices along the guidewire path.
